(19) United States
(12) Patent Application Publication
(10) Pub. No.: US 2010/0119459 A1
(43) Pub. Date: May 13, 2010

(54) Use of ivabradine as diagonstic agent in the method of coronary angiography by multislice computed tomography

(75) Inventors: Guy Lerebours-Pigeonniere, Levallois Perret (FR); Ariane Dubost-Brama, Paris (FR); Carmen Fleurinck, Schelderode (BE)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignee: LES LABORATOIRES SERVIER, Suresnes Cedex (FR)

(21) Appl. No.: 12/590,269

(22) Filed: Nov. 5, 2009

(30) Foreign Application Priority Data
Nov. 7, 2008 (FR) 08.06225

(51) Int. Cl. A61K 49/04

(52) U.S. Cl. 424/9.44

(57) ABSTRACT

Use of ivabradine, or 3-{3-[{[(7S)-3,4-dimethoxybicyclo[4.2.0]octa-1,3,5-trien-7-yl]-methyl}(methyl)amino]propyl}-7,8-dimethoxy- 1,3,4,5-tetrahydro-2H-3-benzazepin-2-one, of its addition salts with a pharmaceutically acceptable acid and of their hydrates, as a diagnostic agent in the method of coronary angiography by multislice computed tomography.

The present invention relates to use of ivabradine, or 3-{3-[{[(7S)-3,4-dimethoxy-bicyclo [4.2.0]octa-1,3,5-trien-7-yl]methyl}(methyl)amino]propyl}-7,8-dimethoxy-1,3,4,5-tetrahydro-2H-3-benzazepin-2-one, of formula (I):

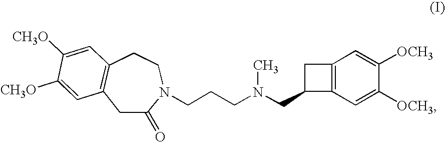

and also its addition salts with a pharmaceutically acceptable acid and hydrates of said addition salts, as a diagnostic agent in the method of coronary angiography by multislice computed tomography.

Ivabradine and also its addition salts with a pharmaceutically acceptable acid, more especially its hydrochloride, and hydrates of said addition salts have very valuable pharmacological and therapeutic properties.

They directly and selectively reduce cardiac pacemaker activity, giving them negative chronotropic properties (reduction of heart rate), without affecting arterial pressure, which makes it possible to consider using them in treating, preventing and improving the prognosis of various cardiovascular diseases associated with myocardial ischaemia such as angina pectoris and myocardial infarction and in chronic heart failure.

The preparation and use in therapeutics of ivabradine and its addition salts with a pharmaceutically acceptable acid, more especially its hydrochloride, have been described in European patent specification EP 0 534 859.

The Applicant has now found that ivabradine and its addition salts, more especially its hydrochloride, have valuable properties allowing their use as diagnostic agents in the method of coronary angiography by multislice computed tomography.

Coronary angiography by multislice computed tomography, or MSCT-CA (MultiSlice Computed Tomography Coronary Angiography), also referred to as MDCT-CA (MultiDetector Computed Tomography Coronary Angiography), is a fast and non-invasive technique making it possible to examine the coronary arteries and to detect, by imaging, coronary disease, especially narrowing (stenosis) or obstruction of the coronary arteries, and also to assess the anatomy and permeability of the vessels and to characterise atheromatous plaques at the tissue level. This method avoids having to use the conventional technique of angiography by cardiac catheterisation, which, owing to its invasive nature, has risks.

In the method of coronary angiography by multislice computed tomography, the patient is injected with an iodinated contrast medium in order to opacify the lumen of the coronary arteries. Image acquisition is then carried out by radiation with X-rays using a multi-row (that is to say, multi-detector) scanner.

The coronary arteries are small-calibre, tortuous, rapidly moving vessels and are therefore difficult to image. Consequently, high spatial and also temporal resolution is required in order to analyse them correctly. The resolution is better, the greater the number of rows. A multi-row scanner generally has from 4 to 64 detectors. The most recent scanners are provided with 64 detectors, and sometimes with a dual X-ray source, which increases the technique's temporal resolution capability.

On the other hand, owing to movement artefacts, image quality is affected by a high heart rate.

The Applicant has now found ivabradine to be capable of lowering the heart rate as a prelude to the procedure. This property makes it possible to consider using ivabradine in patients having a high heart rate and undergoing coronary angiography by multislice computed tomography in order to improve the quality of the images obtained. In addition, as a result of the reduction in heart rate it might be possible to consider reducing the irradiation.

The present invention accordingly relates to the use of ivabradine, of its addition salts with a pharmaceutically acceptable acid and of hydrates of said salts in obtaining compositions for use as diagnostic agents in the method of coronary angiography by multislice computed tomography.

The compositions are in a form suitable for administration by the oral or intravenous route, preferably by the intravenous route.

The useful dosage varies according to the resting heart rate of the person being examined and ranges from 2 to 20 mg per administration.

Administration by the intravenous route is carried out in a bolus or by perfusion.

A bolus is understood to mean rapid administration, lasting preferably less than 30 seconds.

Compositions suitable for administration by the intravenous route can be in the form of an injectable solution or a lyophilisate to be dissolved in a solvent before administration.

The injectable solution is preferably a saline solution.

The concentration of ivabradine base in the injectable solution is preferably from 1 to 5 mg/ml.

The percentage of active ingredient of formula (I) in the injectable solution is preferably from 0.1% to 0.5% by weight.

The percentage of active ingredient of formula (I) in the lyophilisate is preferably from 10% to 50% by weight.

In addition to ivabradine, one of its addition salts with a pharmaceutically acceptable acid or one of the hydrates of one of said addition salts, the compositions suitable for administration by the oral route comprise one or more excipients or carriers such as diluents; lubricants, binders, disintegrating agents, absorbents, colourants, sweeteners.

By way of non-limiting example, there may be mentioned:
as diluents: lactose, dextrose, sucrose, mannitol, sorbitol, cellulose, glycerol,
as lubricants: silica, talc, stearic acid and its magnesium and calcium salts, polyethylene glycol,
as binders: aluminium silicate, magnesium silicate, starch, gelatin, tragacanth, methylcellulose, sodium carboxymethylcellulose and polyvinylpyrrolidone (PVP),
as disintegrating agents: agar, alginic acid and its sodium salt, effervescent mixtures.

The percentage of active ingredient of formula (I) in the composition for administration by the oral route is preferably from 3% to 50% by weight.

### EXAMPLE 1

### Clinical Study. Effect of I.V. Administration of Ivabradine Hydrochloride on Heart Rate in Healthy Volunteers

Resting heart rate (in a lying position) is measured at T0. The subjects are then given an i.v. bolus of a solution of ivabradine hydrochloride containing 16 mg of ivabradine base (treated group, n=8) or of placebo (control group, n=2).

The resting heart rate (in a lying position) is again measured at T0+30 min.

Results:

In the subjects treated with ivabradine, the heart rate is 16% lower than the heart rate in the control group.

### EXAMPLE 2

### Clinical Study. Effect of I.V. Administration of Ivabradine Hydrochloride on Heart Rate in Patients Undergoing Coronary Angiography by Multislice Computed Tomography

The patients selected for this study have a resting heart rate equal to or greater than 70 bpm.

The resting heart rate of the patient is measured at T0.

Patients whose heart rate is from 70 bpm to 79 bpm are given an i.v. bolus of a solution of ivabradine hydrochloride containing 10 mg of ivabradine base (treated group) or of placebo (control group).

Patients whose heart rate is equal to or greater than 80 bpm are given an i.v. bolus of a solution of ivabradine hydrochloride containing 15 mg of ivabradine base (treated group) or of placebo (control group).

The resting heart rate is measured continuously after the bolus injection.

As soon as the heart rate is less than 65 bpm, coronary angiography is carried out on the patient.

The patient is injected with a contrast medium. Image acquisition is then carried out by X-ray radiation using a multi-row scanner having at least 64 detectors.

### EXAMPLE 3

### Injectable Solution Containing 10 mg/5 ml

Formula for the preparation of 1000 ampoules each containing 10 mg of ivabradine base:

| | |
|---|---|
| Ivabradine hydrochloride | 10.78 g |
| Sodium chloride | 45 g |
| Water for injections | 5 litres |

The constituents are mixed together and the resulting solution is distributed into 1000 ampoules each having a capacity of 10 ml.

### EXAMPLE 4

### Injectable Solution Containing 15 mg/7.5 ml

Formula for the preparation of 1000 ampoules each containing 15 mg of ivabradine base:

| | |
|---|---|
| Ivabradine hydrochloride | 16.17 g |
| Sodium chloride | 67.5 g |
| Water for injections | 7.5 litres |

The constituents are mixed together and the resulting solution is distributed into 1000 ampoules each having a capacity of 10 ml.

### EXAMPLE 5

### Lyophilisate for Administration by the Intravenous Route

The constituents of Example 2 are mixed together and the resulting solution is distributed into 1000 ampoules each having a capacity of 10 ml, which are then lyophilised.

### EXAMPLE 6

### Composition for Administration by the Oral Route

Formula for the preparation of 1000 tablets each containing 5 mg of ivabradine base:

| | | |
|---|---|---|
| Ivabradine hydrochloride | 5.39 | g |
| Maize starch | 20 | g |
| Anhydrous colloidal silica | 0.2 | g |
| Mannitol | 63.91 | g |
| Povidone (PVP) | 10 | g |
| Magnesium stearate | 0.5 | g |

1- A method for performing coronary angiography by multislice computed tomography in a subject in need thereof, comprising administering an effective amount of ivabradine, or 3-{3-[{[(7S)-3,4-dimethoxybicyclo[4.2.0]octa-1,3,5-trien-7-yl]-methyl}(methyl)amino]propyl}-7,8-dimethoxy-1,3,4,5-tetrahydro-2H-3-benzazepin-2-one, an addition salt thereof with a pharmaceutically acceptable acid or a hydrate of the addition salt thereof.

2- The method of claim 1, wherein the ivabradine, addition salt thereof, or hydrate of the addition salt thereof, is administered by the intravenous route.

3- The method of claim 2, wherein the ivabradine, addition salt thereof, or hydrate of the addition salt thereof, is administered in the form of an injectable solution.

4- The method of claim 1, wherein the ivabradine, addition salt thereof, or hydrate of the addition salt thereof, is administered by the oral route.
